(19) United States
(12) Patent Application Publication
(10) Pub. No.: US 2024/0074410 A1
(43) Pub. Date: Mar. 7, 2024

(54) Calming Clip for Dogs

(71) Applicant: Seungmin Bang, Los Angeles, CA (US)

(72) Inventor: Seungmin Bang, Los Angeles, CA (US)

(21) Appl. No.: 17/902,911

(22) Filed: Sep. 5, 2022

(51) Int. Cl. A01K 15/00; A61M 21/02

(52) U.S. Cl. CPC A01K 15/00; A61M 21/02; A61M 2021/0077

(57) ABSTRACT

The present invention relates to an advancement in a dog soothing device which provides enhanced functionality and ease of use. The clip at back and the active ingredient are novelty of the proposed invention. The assembly is high quality, easy to use and sustainable in nature.

### BACKGROUND

### Field of the Invention

The present invention pertains to a system for calming and soothing a pet, such as a dog or cat. More particularly, this invention relates to the provision of an apparatus which is in form of clip and has pheromone for soothing the dog.

### Description of the Related Art

It is known that canines and other animals become hyperactive during a long travel or being left unattended in a vehicle. Additionally, puppies may become disoriented and tend to bark after coining to a new home and being alone or separated from its mother. When puppies are separated from their litter, or their birthmother, separation anxiety can become a major problem. The change in environment can put a lot of stress on a young puppy. At bed time, puppies whine when they should be sleeping and keep everyone awake. Sometimes older dogs don't like to be left alone or are afraid of thunder.

Efforts have been expended to develop solutions to help dogs overcome anxiety, restore calm and sleep peacefully.

It is also known that dogs are pack animals and instinctively calm down when they are near another beating heart. A pulsating device to reproduce the beating heart is commercially available such as is sold under the name SnugglePuppie Heart Beat.

In this regard, certain plush stuffed animal toys, particularly adapted for dogs and cats, can include a pulsating device disposed in an interior pouch, which simulates a heartbeat. When the animal engages the stuffed toy, i.e. lies down next to or on the toy and senses the pulsation, the pulsing calms the animal.

In U.S. Provisional Application Ser. No. 61/820,435, filed May 7, 2013, for “Animal Harness,” the entire disclosure of which, including the drawings, is hereby incorporated by reference, discloses an animal harness which includes a pouch that receives and positions a pulsating heartbeat device in contact with the chest of animal for calming the animal during travel and while resting in a vehicle.

The pulsing heartbeat device described hereinabove is believed to have been a positive development in addressing and keeping an animal calm.

The above information and publication is identified herein in recognition of a duty of disclosure of related subject matter, which may be relevant under 37 CFR 1.56, and specifically incorporated herein by reference as regards to the conventional approaches and constructions taught therein.

Yet, there is an ongoing need to provide additional arrangements for calming and soothing canines and other animals, especially a young pet separated from its birthmother and brought to the pet owner's home. The loneliness of such separation can present problems.

There are multiple solutions that have been presented in prior art. However, these solutions are limited and restricted to their conventional architecture, installation system and have considerable shortcomings which adversely affect the convenience with which they can be used. The prior systems have certain limitations including the design of assemblies which make them not suitable for every type of user. Moreover, the ease-of-use ability of these assemblies is also questionable. The current invention proposes an advance structure of dog's soothing device having pheromone as an active ingredient and is easily attached to the animal with a clip structure.

None of the previous inventions and patents, taken either singly or in combination, is seen to describe the instant invention as claimed. Hence, the inventor of the present invention proposes to resolve and surmount existent technical difficulties to eliminate the aforementioned shortcomings of prior art.

### SUMMARY

In light of the disadvantages of the prior art, the following summary is provided to facilitate an understanding of some of the innovative features unique to the present invention and is not intended to be a full description. A full appreciation of the various aspects of the invention can be gained by taking the entire specification, claims, drawings, and abstract as a whole.

The present invention seeks to improve upon prior animal soothing system and provide an enhanced system to perform the designated functionality.

Another object of the present invention is to provide animal soothing system which has clip on its back for secure and ease in attaching to the pet.

It is a further objective of the present invention to provide an improved structure made of high quality materials including but not limited to hard plastic, or other synthetic material which maintain their effectiveness over longer period of time.

It is also the objective of invention to provide dogs soothing device which uses pheromone as an active ingredient and is used to sooth the animal.

Additionally, while discussed in terms of a dog, the invention extends to use in soothing or calming a cat as well as other domestic and domesticated animals.

Thus, it is the objective to provide a new and improved form of an dog soothing device. Other aspects, advantages and novel features of the present invention will become apparent from the detailed description of the invention when considered in conjunction with the accompanying drawings.

This Summary is provided merely for purposes of summarizing some example embodiments, so as to provide a basic understanding of some aspects of the subject matter described herein. Accordingly, it will be appreciated that the above-described features are merely examples and should not be construed to narrow the scope or spirit of the subject matter described herein in any way. Other features, aspects, and advantages of the subject matter described herein will become apparent from the following Detailed Description, Figures, and Claims.

### BRIEF DESCRIPTION OF DRAWING

non-limiting and non-exhaustive embodiments of the present invention are described with reference to the following drawings. The system and method of the present invention will now be described with reference to the accompanying flow chart drawing figure, in which:

FIG. 1 shows the exploded view of assembly as per preferred embodiments of the invention.

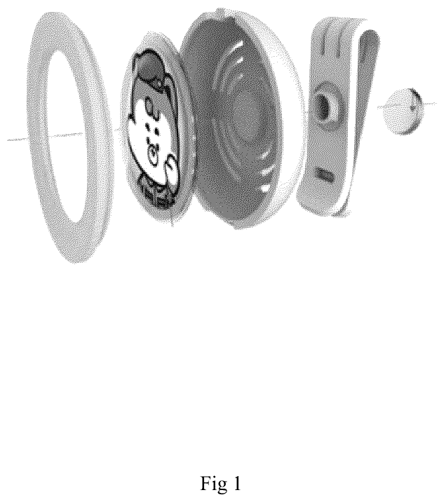

FIG. 2 illustrates multiple views of assembly as per preferred embodiments of the invention.

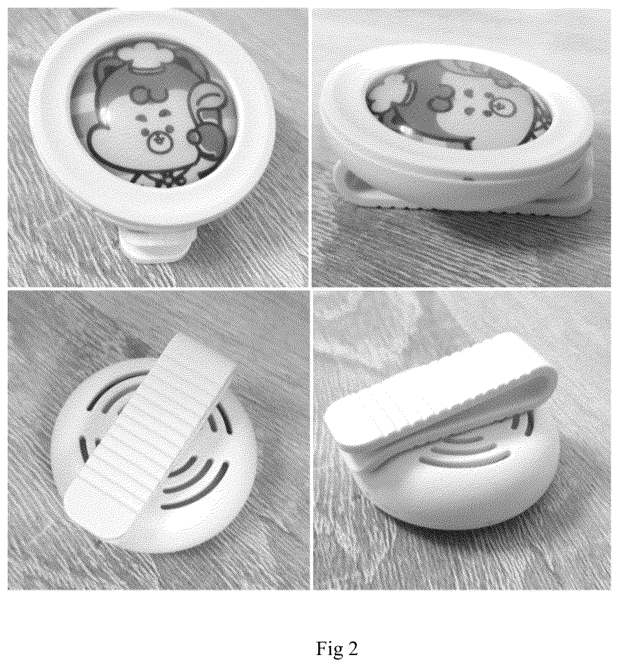

FIG. 3 illustrates perspective use of assembly as per preferred embodiments of the invention.

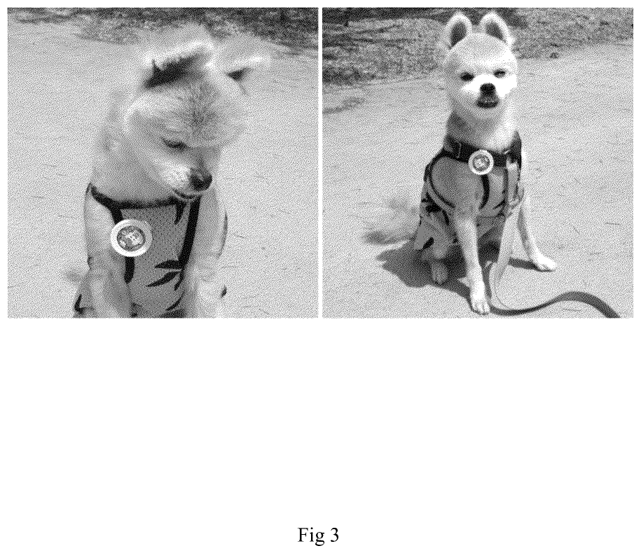

FIG. 4 illustrates another perspective use of assembly as per preferred embodiments of the invention.

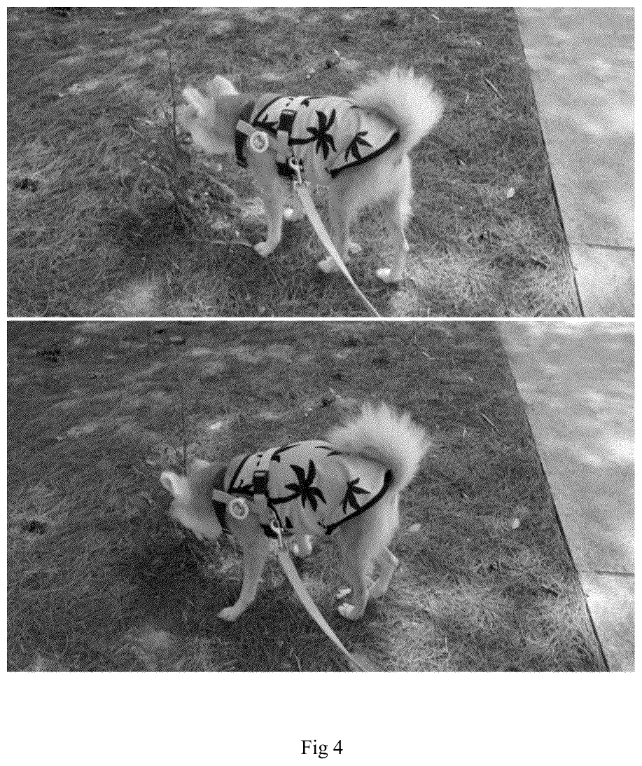

Skilled artisans will appreciate that elements in the figures are illustrated for simplicity and clarity and have not necessarily been drawn to scale. For example, the dimensions of some of the features in the figures may be exaggerated relative to other elements to improve understanding of embodiments of the present invention. The apparatus and method components have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

### DETAILED DESCRIPTION

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

In one aspect of the present invention, the current invention discloses an advance structure of dog soothing device which is in form of clip and can be attached to dog's collar or harness easily.

The assembly as per its preferred embodiments consist of multi layered structure where the top most layer is a plastic case. Under the plastic case the printed cover can be seen. Behind the cover the pouch containing the pheromone as an active ingredient used to soothe the animal is used. The pouch is secured with back cover. The back cover has an associated clip which can be used to attach to dog's collar or harness with ease.

The assembly as per its further embodiments is developed from high quality material preferably plastic and doesn't irritate the animal. It maintains its effectiveness and shape over longer period of use.

While a specific embodiment has been shown and described, many variations are possible. With time, additional features may be employed. The particular shape or configuration of the platform or the interior configuration may be changed to suit the system or equipment with which it is used.

Having described the invention in detail, those skilled in the art will appreciate that modifications may be made to the invention without departing from its spirit. Therefore, it is not intended that the scope of the invention be limited to the specific embodiment illustrated and described. Rather, it is intended that the scope of this invention be determined by the appended claims and their equivalents.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

We claim:

1. An enhanced structure of dog soothing device comprising:
a frontal plastic case;
a printed cover;
a pouch;
a back case; and,
a clip.
The assembly as per claim 1, wherein provides traditional functionality of soothing an animal;
The assembly as per claim 1, wherein uses pheromone as an active ingredient;
The assembly as per claim 1, is developed from high quality plastic; and,
The assembly as per claim 1, is easy to attach or detach.
